(19) United States
(12) Patent Application Publication
(10) Pub. No.: US 2020/0061047 A1
(43) Pub. Date: Feb. 27, 2020

(54) NEW USE OF RIFAMYCIN-QUINOLIZIDONE DUAL-ACTION MOLECULE

(71) Applicant: TenNor Therapeutics Limited, Jiangsu (CN)

(72) Inventors: Zhenkun MA, Jiangsu (CN); Ying YUAN, Jiangsu (CN); Yu LIU, Jiangsu (CN); Xiaomei WANG, Jiangsu (CN)

(73) Assignee: TenNor Therapeutics Limited, Jiangsu (CN)

(21) Appl. No.: 16/488,937

(22) PCT Filed: Feb. 22, 2018
(86) PCT No.: PCT/CN2018/076968

§ 371 Date: Aug. 26, 2019

(30) Foreign Application Priority Data
Feb. 28, 2017 (CN) 201710109969.6

(51) Int. Cl. A61K 31/4545

(52) U.S. Cl. CPC A61K 31/4545

(57) ABSTRACT

A method of inhibiting gastrointestinal ammonia-producing bacteria includes administering to a patient in need thereof a rifamycin-quinolizidone dual-action molecule shown in formula I. The rifamycin-quinolizidone dual-action molecule shown in formula I of present invention has an antibacterial spectrum similar to that of rifaximin, stronger antibacterial activity against the gastrointestinal common ammonia-producing bacteria, low frequency for resistance development, and potential use in prevention and treatment of hepatic encephalopathy and related bacterial infections.

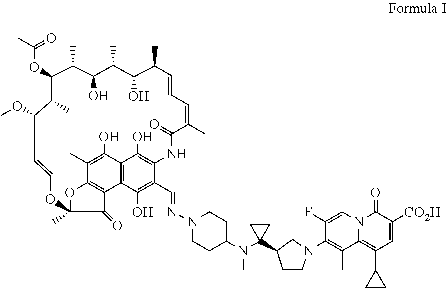 [SMILES: CO[C@H]1/C=C/O[C@@]2(C)Oc3c(C)c(O)c4c(O)c(c(/C=N/N5CCC(N(C)C6([C@@H]7CCN(c8c(F)cn9c(=O)c(C(=O)O)cc(C%10CC%10)c9c8C)C7)CC6)CC5)c(O)c4c3C2=O)NC(=O)/C(C)=C\C=C\[C@H](C)[C@H](O)[C@@H](C)[C@@H](O)[C@@H](C)[C@H](OC(C)=O)[C@@H]1C]

### BACKGROUND

### Technical Field

The present invention belongs to the field of medical chemistry, and particularly relates to a new use of a rifamycin-quinolizidone dual-action molecule.

### Description of Related Art

As one of the important complications of acute or chronic end-stage liver disease and cirrhosis, Hepatic Encephalopathy (HE) seriously affects the prognosis and living quality of patients. For patients with chronic liver disease, once HE occurs, the 1-year survival rate does not exceed 50%, and the 3-year survival rate does not exceed 25%. A patient with Minimal Hepatic Encephalopathy (MHE), i.e. Covert Hepatic Encephalopathy (CHE), often has no clinically significant symptom and MHE can only be found by neuropsychological test. According to statistics, at least 30% of patients with cirrhosis can be accompanied with different degree of HE. According to a recent survey of 16 3A hospitals in 13 provinces and cities in China, it is found that the incidence rate of CHE in hospitalized patients is up to 39.9%, wherein the incidence rate for Child-Pugh Grade A patients is 29.8%, is 39.4% for Child-Pugh Grade B patients, and is 56.1% for Child-Pugh Grade C patients. Patients with CHE are often ignored, and work and live like normal people. However, more and more studies have shown that CHE is the main cause of cognitive dysfunction in patients with cirrhosis, and may affect the living quality and work performance of patients, increase the risk of motor vehicle accident risks, and increase the risk of developing to Overt Hepatic Encephalopathy (OHE). Ammonia poisoning is the major cause of HE/CHE, and over-proliferation of bacteria in the intestinal tract of a patient with cirrhosis, high permeability of the intestinal wall, and intestinal motility disorder jointly cause intestinal bacterial translocation, hyperendotoxemia and hyperammonemia, thereby inducing HE/CHE, increasing liver damage, and forming a vicious circle.

Since ammonia poisoning is the major cause of hepatic encephalopathy, inhibiting the growth of ammonia-producing bacteria, reducing the absorption of ammonia and enhancing the discharge of ammonia are the main means of drug treatment. The main first-line drugs currently recommended for HE/CHE are lactulose and rifaximin, both playing a role by inhibiting intestinal bacteria or improving intestinal micro-ecological structure and reducing intestinal ammonia absorption. However, as an oral non-absorbed disaccharide, lactulose has the adverse effect of abdominal distention, diarrhea or the like, which is difficult to tolerate for many patients. Rifaximin is expensive in price and has a risk of producing drug resistance. Therefore, it is of great significance to develop a HE/CHE treatment drug which has independent intellectual property rights, has a wide antibacterial spectrum for ammonia-producing bacteria, and has better antibacterial activity than rifaximin.

At present, China patent ZL200580031655.4 “rifamycin derivative for treating microbial infections” discloses a compound (R)-3-[(4-{1-[1-(3-carboxy-1-cyclopropyl-7-fluoro-9-methyl-4-oxo-4H-quinolizine-8-yl)-pyrrolidin-3-yl-cyclopropyl]-methylamino}-piperidin-1-ylimino)-methylene]-rifamycin SV which has antimicrobial activity against various bacteria such as Gram positive bacteria, Escherichia coli etc., but has no documented antibacterial activity against gastrointestinal ammonia-producing bacteria.

### SUMMARY

In view of the above defects existing in the prior art, the object of the present invention is to provide a new use of a rifamycin-quinolizidone dual-action molecule which can be effectively against gastrointestinal ammonia-producing bacteria, and can be used to treat hepatic encephalopathy.

The object of the present invention is realized by the following technical solution:

A use of a rifamycin-quinolizidone dual-action molecule shown in formula I against gastrointestinal ammonia-producing bacteria;

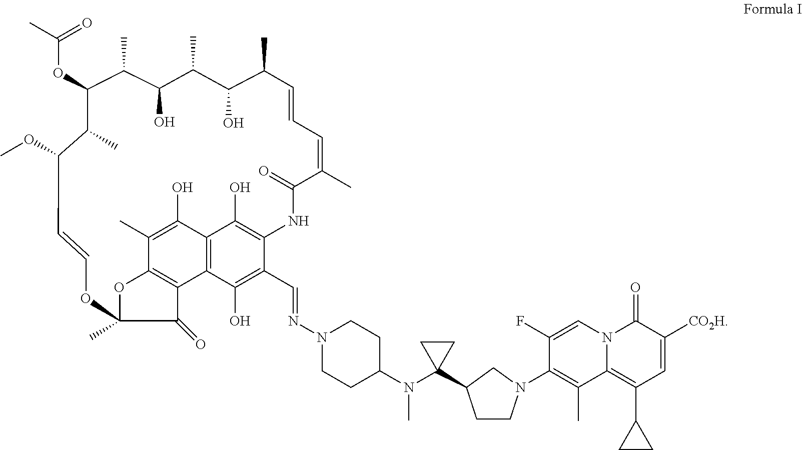 [SMILES: CO[C@H]1/C=C/O[C@@]2(C)Oc3c(C)c(O)c4c(O)c(c(/C=N/N5CCC(N(C)C6([C@@H]7CCN(c8c(F)cn9c(=O)c(C(=O)O)cc(C%10CC%10)c9c8C)C7)CC6)CC5)c(O)c4c3C2=O)NC(=O)/C(C)=C\C=C\[C@H](C)[C@H](O)[C@@H](C)[C@@H](O)[C@@H](C)[C@H](OC(C)=O)[C@@H]1C]

Preferably, in the use, the gastrointestinal ammonia-producing bacteria include one or a combination of more of Bifidobacterium infantis subsp. Infantis, Bacteroides bifidum, Clostridium difficile, Clostridium perfringens, Eggerthella lenta, Escherichia coli, Helicobacter pylori, Lactobacillus salivarius, Fusobacterium necrophorum, Peptostreptococcus prevotii, Morganella morganii, Proteus vulgaris, Salmonella spp and Yersinia enterocolitica.

The present invention further provides a use of the rifamycin-quinolizidone dual-action molecule in preparing a drug for treating Hepatic Encephalopathy (HE) caused by imbalance of gastrointestinal ammonia-producing bacteria.

The present invention further provides a use of the rifamycin-quinolizidone dual-action molecule in preparing a drug for treating Covert Hepatic Encephalopathy (CHE) caused by imbalance of gastrointestinal ammonia-producing bacteria.

Preferably, in the use, the human effective dose of the rifamycin-quinolizidone dual-action molecule is 10-10000 mg per day, and the treatment period is at least 2 days.

Preferably, in the use, the administration route used includes one or a combination of injection administration, oral administration, intracavitary administration, enteral administration, and transdermal absorption.

Preferably, in the use, the administration dosage form used by the use includes one or a combination of injection, suppository, tablet, capsule, patch and extended release dosage form.

The present invention has the prominent effects: the rifamycin-quinolizidone dual-action molecule shown in formula I of the present invention has an antibacterial spectrum similar to that of rifaximin, has stronger antibacterial activity against the common gastrointestinal ammonia-producing bacteria, has the characteristic of low drug resistance frequency, and has potential use in prevention and treatment of hepatic encephalopathy.

In order to make the technical solution of the present invention easier to understand and master, the specific embodiments of the present invention will be described in further detail below with reference to the examples.

### DESCRIPTION OF THE EMBODIMENTS

The present invention is further described below by way of specific embodiments. However, the present invention is not limited to the specific embodiments. The experimental methods described in the following embodiments are conventional methods unless otherwise specified; and the reagents and materials are commercially available unless otherwise specified.

### Embodiment 1

This embodiment provides a use of a rifamycin-quinolizidone dual-action molecule shown in formula I against gastrointestinal ammonia-producing bacteria;

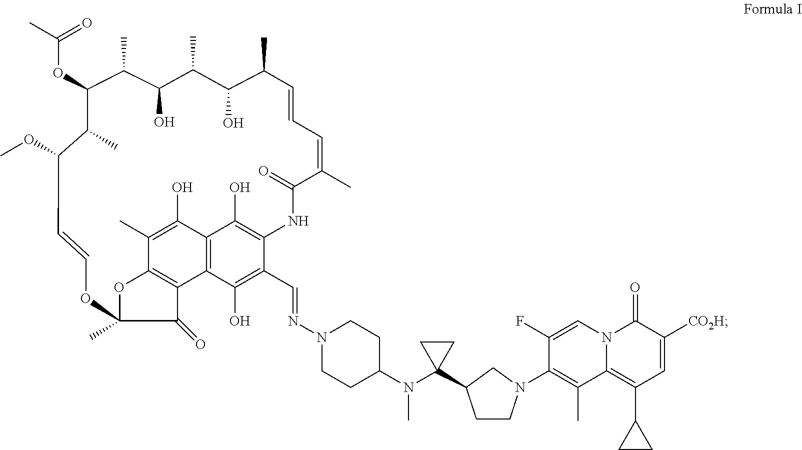 [SMILES: CO[C@H]1/C=C/O[C@@]2(C)Oc3c(C)c(O)c4c(O)c(c(/C=N/N5CCC(N(C)C6([C@@H]7CCN(c8c(F)cn9c(=O)c(C(=O)O)cc(C%10CC%10)c9c8C)C7)CC6)CC5)c(O)c4c3C2=O)NC(=O)/C(C)=C\C=C\[C@H](C)[C@H](O)[C@@H](C)[C@@H](O)[C@@H](C)[C@H](OC(C)=O)[C@@H]1C]

wherein the gastrointestinal ammonia-producing bacteria include one or a combination of more of Bifidobacterium infantis subsp. Infantis, Bacteroides bifidum, Clostridium difficile, Clostridium perfringens, Eggerthella lenta, Escherichia coli, Helicobacter pylori, Lactobacillus salivarius, Fusobacterium necrophorum, Peptostreptococcus prevotii, Morganella morganii, Proteus vulgaris, Salmonella spp and Yersinia enterocolitica.

In this embodiment, compound I-rifamycin-quinolizidone dual-action molecule is used in a drug sensitivity test on pathogenic bacteria associated with hepatic encephalopathy, the pathogenic bacteria including the above ammonia-producing bacteria. Except that Haemophilus is tested using the microscale broth dilution method, all the other bacteria are tested using the agar dilution method consistent with that in the Guideline of the Clinical and Laboratory Standards Institute (CLSI; 1-3). All the other drug susceptibility tests are performed under anaerobic conditions except that some selective isolates are tested under both aerobic and anaerobic conditions. Control compounds include metronidazole, rifampicin, clindamycin (under anaerobic conditions) and ciprofloxacin (under aerobic and anaerobic conditions).

Material and Method

Test Compounds

Provided by TenNor Therapeutics Ltd., and stored at −20° C. before test. Three control drugs are provided by Sigma. All stock liquors are allowed to stand for at least 1 hour before being automatically sterilized.

Test Strains

The tested clinical isolates may be reference strains obtained from American Type Culture Collection, ATCC, Manassas, Va. After being received, the strains are respectively inoculated on appropriate agar plates and placed under optimized conditions for growth. The growing strains are cloned in the broth containing cryoprotectant to prepare bacterial suspensions, and the bacterial suspensions are subpackaged and then stored in freezing at −80° C. Before test, the frozen bacteria are inoculated into appropriate agar dishes and cultured for growth. Anaerobic bacteria grow for 48 hours at 35° C. in a Bactron II oxygen-free cabinet (Shel Lab, Cornelius, Oreg.) before test.

Test Broths

The broth used for drug sensitivity test by the anaerobic agar dilution method is supplementary Brucella Agar (SBA) composed of Brucella agar containing 5 μg/mL of hemin (BD/BBL; Art. No.: 5300551), 1 μg/mL of vitamin K1 (Sigma, St. Louis, Mo.; Art. No.: SLBC4685V) and 5% lake sheep blood (Cleveland Scientific, Bath, Ohio; Art. No.: 291958).

Mueller Hinton Agar (MHA; Becton Dickinson, Sparks, Md.; Art. No.: 6229829) is used to perform drug sensitivity test by the aerobic agar dilution method. 5% of lake sheep red blood cell is added when testing streptococcus.

Haemophilus Test Medium (HTM, Teknova, Hollister, Calif.; Art. No.: 895120) is used to perform drug sensitivity test on Hemophilus using the microscale broth dilution method under aerobic and anaerobic conditions.

Preparation and storage of all the above broths are performed in accordance with CLSI (1-3).

The Minimum Inhibitory Concentration (MIC) is determined using the agar dilution method.

The MIC values of all microorganisms except Haemophilus are determined using the agar dilution method in the CLSI (1-2). Drugs are manually diluted and drug-containing agar plates are prepared in accordance with the CLSI guideline (1-2). To dry the agar surface, a multi-well plate is plated at room temperature for 1 hour. The agar plate used for testing under anaerobic condition is pre-placed in an oxygen-free cabinet for about 1 hour. All isolates are adjusted to 0.5 McFarland Standard in appropriate broths using a nephelometer (Dade Behring MicroScan, Wet Sacramento, Calif.). Then, each bacterial suspension is transferred into wells of the test plate using a stainless steel duplicator. About 10⁵/1−2 microliters of bacteria are inoculated on the agar surface of each well, and after drying, the drug plate and the drug-free control plate are placed in the oxygen-free cabinet to be cultured in an anaerobic environment at 35° C. for 42-48 hours, and cultured in an aerobic environment at 35° C. for 24-48 hours. After culturing, the MIC is determined in accordance with the CLSI guideline (1-2).

The test results are shown in Tables 1-2.

TABLE 1

| | | MIC (μg/mL) | | | |
|---|---|---|---|---|---|
| Strain Name | No. | Compound I | Ciprofloxacin | Rifaximin | Metronidazole |
| Bifidobacterium | ATCC 15702 | 2 | 2 | 0.25 | 16 |
| infantis subsp. Infantis | | | | | |
| Bacteroides bifidum | ATCC 1569 | 16 | 2 | 0.5 | 8 |
| Clostridium difficile | ATCC 700057 | 0.004 | 16 | 0.004 | 0.5 |
| Clostridium | ATCC 13124 | 0.03 | 0.5 | 0.12 | 2 |
| perfringens | | | | | |
| Eggerthella lenta | ATCC 43055 | 0.06 | 1 | 0.12 | 1 |
| Fusobacterium | ATCC 25286 | 0.12 | 1 | 16 | 0.12 |
| necrophorum | | | | | |
| Escherichia coli | ATCC 25922 | 0.12 | 0.008 | 8 | Un-tested |
| Helicobacter pylori | ATCC 700824 | 0.12 | 0.12 | 2 | Un-tested |
| Lactobacillus | JMI 941623 | 0.5 | 1 | 1 | Un-tested |
| salivarius | | | | | |
| Peptostreptococcus | ATCC 9321 | 0.03 | 1 | 0.03 | 256 |
| prevotii | | | | | |
| Klebsiella oxytoca | JMI 933995 | 4 | 0.015 | 32 | Un-tested |
| Morganella morganii | MMX 6123 | 16 | 1 | 16 | Un-tested |
| Proteus mirabilis | JMI 933599 | 8 | 0.0015 | 8 | Un-tested |
| Proteus vulgaris | MMX 6454 | 2 | 0.12 | 4 | Un-tested |
| Salmonella spp | ATCC 35987 | 0.06 | 0.015 | 16 | Un-tested |
| Yersinia enterocolitica | ATCC 23715 | 0.12 | 0.015 | 8 | Un-tested |

TABLE 2

| | | MIC (μg/mL) | | | | | | | |
|---|---|---|---|---|---|---|---|---|---|
| | | Compound I | | Rifampicin | | Ciprofloxacin | | Metronidazole | |
| Strain Name | No. | Aerobic | Anaerobic | Aerobic | Anaerobic | Aerobic | Anaerobic | Aerobic | Anaerobic |
| Actinomyces naeslundii | JMI | 0.03 | Un- | <0.002 | Un- | 2 | Un- | Un- | Un- |
| | 534283 | | tested | | tested | | tested | tested | tested |
| Alcaligenes faecalis | JMI | 32 | Un- | 16 | Un- | 1 | Un- | Un- | Un- |
| | 940880 | | tested | | tested | | tested | tested | tested |
| Bacteroides vulgatus | JMI | 16 | Un- | 0.06 | Un- | >16 | Un- | Un- | Un- |
| | 927011 | | tested | | tested | | tested | tested | tested |
| Bordetella avium | ATCC | 4 | >64 | 16 | 64 | 1 | 2 | >256 | >256 |
| | 6945 | | | | | | | | |
| Corynebacterium urealyticum | JMI | 0.03 | Un- | 0.004 | Un- | >16 | Un- | Un- | Un- |
| | 2015_30 | | tested | | tested | | tested | tested | tested |
| Serratia marcescens | ATCC | 4 | >64 | 64 | 64 | 0.12 | 0.25 | >256 | >256 |
| | 43862 | | | | | | | | |
| Proteus vulgaris | ATCC | 0.5 | >64 | 16 | 8 | 0.03 | 0.03 | >256 | >256 |
| | 1331 | | | | | | | | |
| Enterobacter aerogenes | ATCC | 1 | >64 | 64 | 64 | 0.03 | 2 | >256 | >256 |
| | 13048 | | | | | | | | |
| Haemophilus parainfluenzae | ATCC | 0.25 | No | 0.5 | No | ≤0.12 | No | 128 | No |
| | 49247 | | growth | | growth | | growth | | growth |
| Haemophilus influenzae | ATCC | 0.12 | 0.12 | 0.25-1 | 0.25 | ≤0.12 | ≤0.12 | 64 | 64 |
| | 49247 | | | | | | | | |
| Staphylococcus saprophyticus | JMI | 0.5 | Un- | 1 | Un- | 1 | Un- | Un- | Un- |
| | 2016_703 | | tested | | tested | | tested | tested | tested |
| Streptococcus parasanguinis | MMX | 0.25 | 8 | 0.03 | 16 | 1 | 1 | >256 | >256 |
| | 5671 | | | | | | | | |
| Streptococcus salivarius | ATCC | 0.5 | >64 | 0.03 | 32 | 1 | 2 | >256 | >256 |
| | 25975 | | | | | | | | |
| Streptococcus pneumoniae | ATCC | 0.06 | 0.06 | 0.03 | 0.12 | 1 | 2 | >256 | 128 |
| | 49619 | | | | | | | | |

It can be seen from the test results in Table 1 that compound I has activity against ammonia-producing bacteria which is identical to or stronger than that of rifaximin or ciprofloxacin. The test results in Table 2 indicate that compound I also has activity against other pathogenic bacteria associated with the microbial of a patient with hepatic encephalopathy reported in literatures, for example, one or a combination of more of Actinomyces naeslundii, Bacteroides vulgatus, Bacteroides fragilis, Bordetella avium, Corynebacterium urealyticum, Enterobacter aerogenes, Haemophilus parainfluenzae, Haemophilus influenzae, Staphylococcus saprophyticus, Proteus vulgaris, Serratia marcescens, Streptococcus parasanguinis, Streptococcus salivarius and Streptococcus pneumoniae.

According to the in vitro antibacterial activity of the compound I, it is predicted that the effective dose thereof is 1/100 of rifaximin, which is equivalent to 10 mg. In order to further improve the drug effect, the dose of the compound I may be increased to 10 g which is the highest effective dose thereof.

### Embodiment 2

This embodiment provides a formula and preparation method for an immediate release oral dosage form of the rifamycin-quinolizidone dual-action molecule shown in formula I.

| Rifamycin-quinolizidone dual-action molecule | 100 | g |
|---|---|---|
| shown in formula I | | |
| Mannitol | 154 | g |
| Sodium starch glycolate | 20 | g |
| Polyvinyl pyrrolidone K30 | 9 | g |
| Sodium dodecyl sulfate | 3 | g |
| Silicon dioxide | 8 | g |
| Magnesium stearate | 6 | g |
| Purified water | Appropriate amount | |
| Prepared in total | 1000 | EA |

Weighing the rifamycin-quinolizidone dual-action molecule shown in formula I and excipients according to the formula; dissolving Polyvinyl Pyrrolidone K30 (PVP K30) and Sodium Dodecyl Sulfate (SDS) in purified water, stirring for 1 hour, and taking the stirred product as binder for later use; sieving the rifamycin-quinolizidone dual-action molecule shown in formula I, mannitol and sodium starch glycolate (DST) with a sieve of 30 meshes, adding the mixture into a granulator for premixing, wherein the impeller stirring speed is 700 rpm, and the time duration is about 15 minutes; using a peristaltic pump to add an appropriate amount of purified water and adhesive into the granulator mixture at a fixed speed (145-165 g/min), wherein the stirring speed of the granulator impeller is 400 rpm, and the time duration is about 1-2 minutes, and continuing to mix for 0.5-1 minute after the adhesive is completely added; drying the wet particles using a fluid bed, supposing that the air inlet temperature is 60° C., and the air inlet rate is 40 m³/h; according to the weight of the dried dry particle material, calculating the weight of silicon dioxide and magnesium stearate to be added, placing the silicon dioxide and dry particles in a bin blender for mixing, wherein the mixing time duration 15 minutes, and the speed is 20 rpm; then adding magnesium stearate, wherein the mixing time duration is 6 minutes at 20 rpm, taking the totally mixed material to fill No. 0 capsules using a capsule filling machine, and then obtaining hard capsules of the rifamycin-quinolizidone dual-action molecule shown in formula I.

Tableting the totally mixed material using a tableting machine, and then obtaining tablets of the rifamycin-quinolizidone dual-action molecule shown in formula I.

### Embodiment 3

This embodiment provides a preparation method for injections of the rifamycin-quinolizidone dual-action molecule shown in formula I.

| | | |
|---|---|---|
| Rifamycin-quinolizidone dual-action molecule | 30 | g |
| shown in formula I | | |
| Mannitol | 20 | g |
| Sodium formaldehyde sulfoxylate | 0.5 | g |
| Tween-80 | 0.1 | g |
| 1N NaOH | 36 | mL |

| | |
|---|---|
| Water for injection | Added to 1000 mL |

Adding mannitol, sodium formaldehyde sulfoxylate and Tween-80 into an appropriate amount of water for injection under the protection of nitrogen, adding the rifamycin-quinolizidone dual-action molecule shown in formula I, stirring for 10-15 minutes at the intermediate speed, wetting the rifamycin-quinolizidone dual-action molecule shown in formula I, slowing adding IN NaOH dropwise, wherein about 175 minutes are consumed (rapid at first and slow down then), until the rifamycin-quinolizidone dual-action molecule shown in formula I is completely dissolved, filtering using two microporous membranes of 0.45+0.22 μm, filling the filtrate into 10 mL glass bottles, each bottle being filled with 3.5 mL, transferring the glass bottles into a freeze dryer for freeze-drying, and obtaining freeze-dried powder for injections of the rifamycin-quinolizidone dual-action molecule shown in formula I after screwing caps.

### Embodiment 4

This embodiment provides a preparation method for enteric controlled release preparations of the rifamycin-quinolizidone dual-action molecule shown in formula I.

Formula of Drug-Containing Granules

| Rifamycin-quinolizidone dual-action molecule | 2 | g |
|---|---|---|
| shown in formula I | | |
| Starch | 80 | g |
| Mannitol | 20 | g |
| Carboxymethyl starch sodium | 4 | g |
| Sodium dodecyl sulfate | 2 | g |
| Formula of protective layer: | | |
| Mannitol | 50 | g |
| Cane sugar | 8 | g |
| Hydroxypropyl methylcellulose | 3.2 | g |
| Enteric coating layer | | |
| Hydroxypropyl methylcellulose phthalate (HPMCP) | 32 | g |
| Talc | 1.86 | g |

Taking 80 g of starch, 2 g of rifamycin-quinolizidone dual-action molecule shown in formula I, 20 g of mannitol, 4 g of carboxymethyl starch sodium (CMS) and 2 g of Sodium dodecyl sulfate for dry mixing, preparing 4% hydroxypropyl methylcellulose phthalate (HPMCP) solution and mixing the solution with 95% ethanol in a proportion (2:8) as binder, and preparing drug-containing granules;

dissolving 50 g of mannitol into the remaining HPMCP solution, coating on the surface of drug-containing granules, mixing syrup of formula amount with 95% ethanol in a certain proportion (44:56), and spraying the mixture on the granule surface to be used as a protective layer;

finally, mixing 7.5% HPMCP of prescription amount with 95% ethanol in a certain proportion (80:20), and coating on the granule surface as an enteric coating layer; and

performing granule drying, granulating, and tableting, and obtaining the small intestine specific controlled release tablets of the rifamycin-quinolizidone dual-action molecule shown in formula I.

It can be seen from the data that the rifamycin-quinolizidone dual-action molecule (formula I) of the present invention has antibacterial activity against common gastrointestinal ammonia-producing bacteria, has the characteristic of low drug resistance frequency, and has a significant treatment effect on hepatic encephalopathy and/or covert hepatic encephalopathy.

1. A method of inhibiting gastrointestinal ammonia-producing bacteria, comprising:
administering to a patient in need thereof a rifamycin-quinolizidone dual-action molecule shown in formula I;

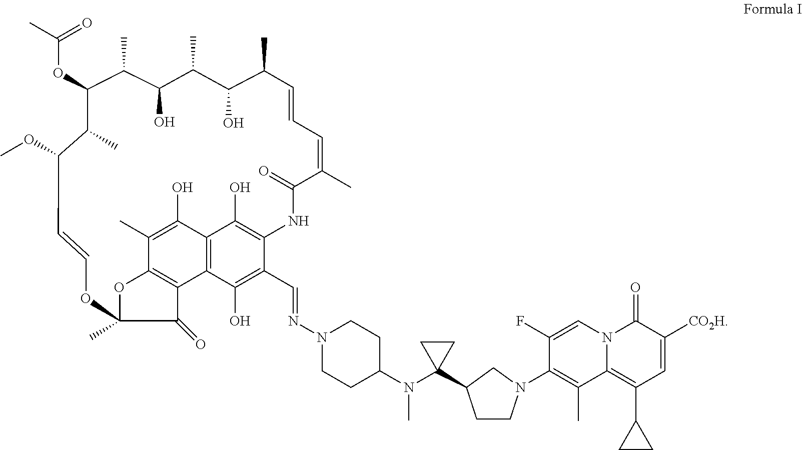 [SMILES: CO[C@H]1/C=C/O[C@@]2(C)Oc3c(C)c(O)c4c(O)c(c(/C=N/N5CCC(N(C)C6([C@@H]7CCN(c8c(F)cn9c(=O)c(C(=O)O)cc(C%10CC%10)c9c8C)C7)CC6)CC5)c(O)c4c3C2=O)NC(=O)/C(C)=C\C=C\[C@H](C)[C@H](O)[C@@H](C)[C@@H](O)[C@@H](C)[C@H](OC(C)=O)[C@@H]1C]

2. The method according to claim 1, wherein the gastrointestinal ammonia-producing bacteria comprises one or a combination of Bifidobacterium infantis subsp. Infantis, Bacteroides bifidum, Clostridium difficile, Clostridium perfringens, Eggerthella lenta, Escherichia coli, Helicobacter pylori, Lactobacillus salivarius, Fusobacterium necrophorum, Peptostreptococcus prevotii, Morganella morganii, Proteus vulgaris, Salmonella spp and Yersinia enterocolitica.

3. A method of treating hepatic encephalopathy caused by imbalance of gastrointestinal ammonia-producing bacteria, comprising:
administering to a patient in need thereof a pharmaceutical composition comprising the rifamycin-quinolizidone dual-action molecule according to claim 1.

4. A method of treating covert hepatic encephalopathy caused by imbalance of gastrointestinal ammonia-producing bacteria, comprising:
administering to a patient in need thereof a pharmaceutical composition comprising the rifamycin-quinolizidone dual-action molecule according to claim 1.

5. The method according to claim 3, wherein a human effective dose of the rifamycin-quinolizidone dual-action molecule is 10-10000 mg per day, and a treatment period is at least 2 days.

6. The method according to claim 3, wherein an administration route used comprises one or a combination of injection administration, oral administration, intracavitary administration, enteral administration, and transdermal absorption.

7. The method according to claim 3, wherein an administration dosage form used comprises one or a combination of injection, suppository, tablet, capsule, patch and extended release dosage form.

8. The method according to claim 4, wherein a human effective dose of the rifamycin-quinolizidone dual-action molecule is 10-10000 mg per day, and the treatment period is at least 2 days.

9. The method according to claim 4, wherein an administration route used comprises one or a combination of injection administration, oral administration, intracavitary administration, enteral administration, and transdermal absorption.

10. The method according to claim 4, wherein an administration dosage form used comprises one or a combination of injection, suppository, tablet, capsule, patch and extended release dosage form.
